(19) United States
(12) Patent Application Publication
(10) Pub. No.: US 2025/0135057 A1
(43) Pub. Date: May 1, 2025

(54) FLUID STERILIZATION DEVICE

(71) Applicant: TOYODA GOSEI CO., LTD., Kiyosu-shi (JP)

(72) Inventors: Satoshi WADA, Kiyosu-shi (JP); Hisayuki MIKI, Kiyosu-shi (JP)

(21) Appl. No.: 18/929,123

(22) Filed: Oct. 28, 2024

(30) Foreign Application Priority Data
Oct. 30, 2023 (JP) 2023-185336

(51) Int. Cl. A61L 2/10; A61L 2/26; A61L 9/20

(52) U.S. Cl. CPC A61L 2/10; A61L 2/26; A61L 9/20; A61L 2202/11; A61L 2209/12

(57) ABSTRACT

A fluid sterilization device includes: a cylindrical flow path tube having a flow path space therein through which a fluid is capable of flowing; a first inlet provided at a first end of the flow path tube and allowing the fluid to flow into the flow path space; a second inlet provided at a second end of the flow path tube opposite to the first end and allowing the fluid to flow into the flow path space; an outlet provided between the first end and the second end and allowing the fluid flowing through the flow path space to flow out; a first light source unit disposed at the first end and configured to emit ultraviolet light into the flow path space; and a second light source unit disposed at the second end and configured to emit ultraviolet light into the flow path space.

### CROSS-REFERENCE TO RELATED APPLICATIONS

This application is based on and claims priority under 35 USC 119 from Japanese Patent Application No. 2023-185336 filed on Oct. 30, 2023, the entire content of which is incorporated herein by reference.

### TECHNICAL FIELD

The present disclosure relates to a fluid sterilization device.

### BACKGROUND ART

A sterilization device that sterilizes bacteria and viruses in running water by irradiating the water with ultraviolet light is known. A mercury lamp is widely used as a light source. Since the mercury lamp uses mercury and thus the mercury lamp is highly toxic, there is a problem that the mercury lamp has a large environmental load. Further, there is also a problem that use of a mercury lamp increases a size of the sterilization device. Therefore, the mercury lamp has been replaced with an ultraviolet LED.

JP2017-051289A discloses a water sterilization device using an LED that emits ultraviolet light, the water sterilization device includes a flow path tube through which water flows, a water inlet provided at both ends of the flow path tube, a water outlet provided at a center of the flow path tube, and an ultraviolet LED that emits ultraviolet light into the flow path tube from outside of both ends of the flow path tube. In this configuration, since an incident portion of the ultraviolet LED is positioned in a vicinity of each water inlet, the water flowing in is easily irradiated with the ultraviolet light, and a sterilization effect is improved.

### SUMMARY OF INVENTION

However, in the configuration disclosed in JP2017-051289A, heat dissipation measures for the ultraviolet LED are not sufficiently taken, and the ultraviolet LED is at a high temperature and may be damaged when continuously used. Further, providing a separate cooling device for cooling both ultraviolet LEDs provided outside the flow path tube leads to an increase in size and cost of the device.

The present disclosure has been made in view of such a background, and aspect of the present disclosure relate to providing a fluid sterilization device capable of improving a cooling effect and preventing an increase in size and cost.

According to an aspect of the present disclosure, there is provided a fluid sterilization device including: a cylindrical flow path tube having a flow path space therein through which a fluid is capable of flowing; a first inlet provided at a first end of the flow path tube and allowing the fluid to flow into the flow path space; a second inlet provided at a second end of the flow path tube opposite to the first end and allowing the fluid to flow into the flow path space; an outlet provided between the first end and the second end of the flow path tube and allowing the fluid flowing through the flow path space to flow out; a first light source unit disposed at the first end within the flow path space and configured to emit ultraviolet light into the flow path space; and a second light source unit disposed at the second end within the flow path space and configured to emit ultraviolet light into the flow path space.

In the fluid sterilization device according to the above aspect, since the first light source unit and the second light source unit are both positioned in the flow path space, heat dissipation is promoted by bringing into contact with the fluid flowing through the flow path space, and the cooling effect is improved. Therefore, the first light source unit and the second light source unit are prevented from being damaged even when continuously used. Further, since there is no need to provide a separate cooling device for cooling the first light source unit and the second light source unit, an increase in size and cost may be prevented.

### BRIEF DESCRIPTION OF DRAWINGS

Exemplary embodiment(s) of the present invention will be described in detail based on the following figures, wherein:

FIG. 1 is a perspective view of a fluid sterilization device according to Embodiment 1;

FIG. 2 is a cross-sectional view taken along line II-II in FIG. 1;

FIG. 3A is a cross-sectional view taken along line IIIa-IIIa in FIG. 1;

FIG. 3B is a cross-sectional view taken along line IIIb-IIIb in FIG. 1;

FIG. 3C is a cross-sectional view taken along line IIIc-IIIc in FIG. 1;

FIG. 4A is an enlarged view of a vicinity of a first light source unit in FIG. 2;

FIG. 4B is an enlarged view of a vicinity of a second light source unit in FIG. 2;

FIG. 4C is an enlarged view of a central portion of a flow path tube in FIG. 2;

FIG. 5A is a cross-sectional view of each of a first light source unit and a second light source unit taken along a plane of the flow path tube including an axis;

FIG. 5B is an example of a cross-sectional view taken along line Vbc-Vbc in FIG. 5A;

FIG. 5C is another example of a cross-sectional view taken along line Vbc-Vbc in FIG. 5A, according to Embodiment 2;

FIG. 6 is a sectional view of each of a first light source unit and a second light source unit taken along a plane of a flow path tube including an axis, according to Embodiment 3;

FIG. 7 is a sectional view of each of a first light source unit and a second light source unit taken along a plane of a flow path tube including an axis, according to Embodiment 4; and

FIG. 8 is a sectional view of a fluid sterilization device taken along a plane of a flow path tube including an axis, according to Modification 1 of Embodiment 1.

### DESCRIPTION OF EMBODIMENTS

It is preferable that the first inlet is provided on a side wall of the flow path tube at the first end, and a first central axis, which is a central axis of the first inlet, does not intersect with a central axis of the flow path tube when viewed from an extending direction of the central axis of the flow path tube, such that a fluid flowing from the first inlet forms a first helical flow along an inner circumferential surface of the flow path tube, and the second inlet is provided on a side wall of the flow path tube at the second end, and a second central axis, which is a central axis of the second inlet, does not intersect the central axis of the flow path tube when viewed from the extending direction of the central axis of the flow path tube, such that a fluid flowing from the second inlet forms a second helical flow in the same swirling direction as the first helical flow along the inner circumferential surface of the flow path tube.

In this case, the fluid flowing into the flow path space from the first inlet and the fluid flowing from the second inlet into the flow path space flow through the flow path space along the helical flows in the same swirling direction. Accordingly, by smoothing the flow of fluid in the flow path space while lengthening residence time in the flow path space of the fluid flowing through the flow path space, the cooling effect of the first light source unit and the second light source unit may be improved, thereby improving the sterilization effect of the fluid and optimizing the sterilization effect for continuous use.

It is preferable that the flow path tube has a straight tube shape, the first light source unit and the second light source unit are disposed to face each other, the first light source unit is positioned at a position intersecting with the first central axis of the first inlet when viewed from the extending direction of the central axis of the flow path tube and a direction orthogonal to the first central axis, and the second light source unit is positioned at a position intersecting with the second central axis of the second inlet when viewed from the extending direction of the central axis of the flow path tube and a direction orthogonal to the second central axis. In this case, the fluid may be brought into contact with the first light source unit immediately after flowing into the flow path space from the first inlet, and may be brought into contact with the second light source unit immediately after flowing into the flow path space from the second inlet. Accordingly, the cooling effect of the first light source unit and the second light source unit may be further improved.

It is preferable that the first light source unit includes a light emitting element that emits ultraviolet light, a support portion provided to protrude from an end surface of the first end of the flow path tube inside the flow path space toward the second end, and an accommodation portion that is provided at a front end of the support portion and accommodates the light emitting element, the accommodation portion is formed to extend from the front end of the support portion to an outside of the support portion in a radial direction over an entire periphery of the front end of the support portion, and the second light source unit has the same configuration as the first light source unit, and the configuration is reversed in a direction facing the first light source unit.

In this case, since the accommodation portion is formed to extend from the front end of the support portion to the outside of the support portion in the radial direction over the entire periphery of the front end of the support portion in the first light source unit and the second light source unit, the fluid may be brought into contact with an inner surface of the accommodation portion (surface closer to the support portion), and the accommodation portion may be efficiently cooled.

The accommodation portion in each of the first light source unit and the second light source unit may have a peripheral wall that is formed on an outer surface of the accommodation portion closer to the support portion, protrudes toward the support portion, and surrounds at least a portion of the front end of the support portion. In this case, in the first light source unit and the second light source unit, the fluid may be retained on the inner surface of the accommodation portion (surface closer to the support portion) by the peripheral wall, and the accommodation portion may be more efficiently cooled by the fluid.

The peripheral wall in each of the first light source unit and the second light source unit may be positioned outside the light emitting element when viewed from a central axis direction of the support portion. In this case, in the first light source unit and the second light source unit, the fluid may be retained in a region of the inner surface of the accommodation portion directly below the light emitting element, and the accommodation portion may be more efficiently cooled by the fluid.

It is preferable that the support portion in each of the first light source unit and the second light source unit has a truncated cone-shaped portion whose diameter decreases toward the front end of the support portion in a protruding direction. In this case, the fluid flowing into the flow path space in the flow path tube from the first inlet and the second inlet hits a side surface of the support portion having an inclination in the truncated cone-shaped portion and is reflected in the protruding direction of the support portion, forming a flow path toward the accommodation portion. Therefore, the fluid may be efficiently brought into contact with the accommodation portion, and cooling efficiency may be improved. Further, heat of the accommodation portion may be efficiently diffused to the support portion, and the support portion may be efficiently cooled by the fluid.

The support portion in each of the first light source unit and the second light source unit may have a cylindrical column shape.

A surface of the accommodation portion in each of the first light source unit and the second light source unit closer to the support portion may be provided with a spiral groove or a protrusion portion. Further, a surface of the accommodation portion in each of the first light source unit and the second light source unit closer to the support portion may be provided with a radial groove or a protrusion portion.

### Embodiment 1

### 1. Overview of Configuration of Fluid Sterilization Device 1

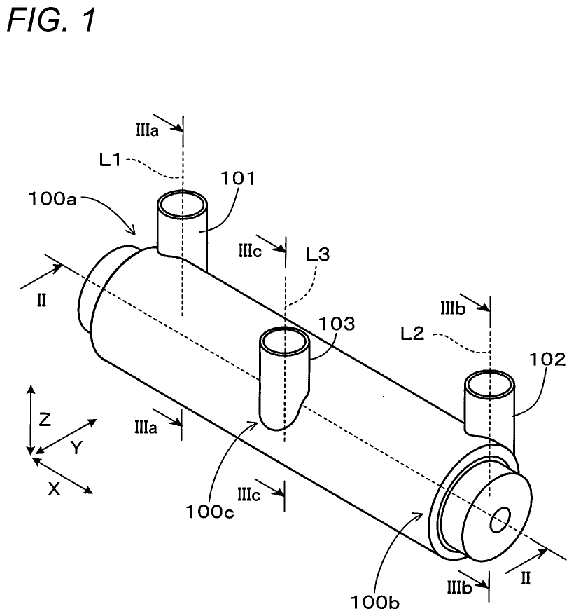

FIG. 1 is a view schematically illustrating a configuration of a fluid sterilization device 1 according to Embodiment 1. As illustrated in FIG. 1, the fluid sterilization device 1 according to Embodiment 1 includes a flow path tube 100 and two light source units 110 (110a, 110b). Further, the light source unit 110 includes LED packages 140, a support portion 120, and an accommodation portion 130.

The fluid sterilization device 1 according to Embodiment 1 is a device that causes a fluid to flow from a first inlet 101 and a second inlet 102 of the flow path tube 100 to a flow path space inside the flow path tube 100, irradiates the fluid with ultraviolet light from the light source unit 110 to sterilize the fluid, and discharges the sterilized fluid from an outlet 103. The fluid to be sterilized may be a gas or a liquid, and may be a mixture of a gas and a liquid, a mixture of a gas and a powdery solid, or the like as long as the fluid has fluidity. When the fluid is a liquid, examples thereof include water, oil, alcohol, and a solution containing the same as a solvent.

### 2. Details of Each Configuration of Fluid Sterilization Device 1

Next, each configuration of the fluid sterilization device 1 will be described in detail.

### 2-1. Configuration of Flow Path Tube 100

The flow path tube 100 has a cylindrical tube shape and has a cylindrical column-shaped space therein. This space is a flow path space through which the fluid to be sterilized flows. In Embodiment 1, an extending direction of a central axis O of the flow path tube 100 is defined as a longitudinal direction X, one radial direction of the flow path tube 100 is defined as a width direction Y, and a direction orthogonal to the longitudinal direction X and the width direction Y is defined as a height direction Z. Both ends of the flow path tube 100 in the longitudinal direction X are each provided with a light source unit 110. Further, the first inlet 101 is provided on a side wall of a first end 100a of the flow path tube 100 in the longitudinal direction X, the second inlet 102 is provided on a side wall of a second end 100b of the flow path tube 100 in the longitudinal direction X, and the outlet 103 is provided on a side wall of a central portion 100c between the first end 100a and the second end 100b of the flow path tube 100 in the longitudinal direction X.

Examples of a material for the flow path tube 100 include SUS, titanium, and polytetrafluoroethylene (PTFE). An inner wall surface of a resin material resistant to ultraviolet light may be covered with a material having a high reflectance to ultraviolet light. The resin material having resistance to the ultraviolet light is, for example, vinyl chloride. Further, examples of the material having a high reflectance to the ultraviolet light include aluminum and PTFE. Further, an outer wall surface of a material that transmits ultraviolet light may be covered with a material having a high reflectance to ultraviolet light. Examples of the material that transmits the ultraviolet light include sapphire, ultraviolet-transmitting glass, fluororesin, and acrylic resin. An inner wall surface of the flow path tube 100 preferably has an arithmetic mean roughness Ra of 0.2 nm to 10 μm. A resistance of the inner wall surface decreases, allowing the flow to be easily maintained.

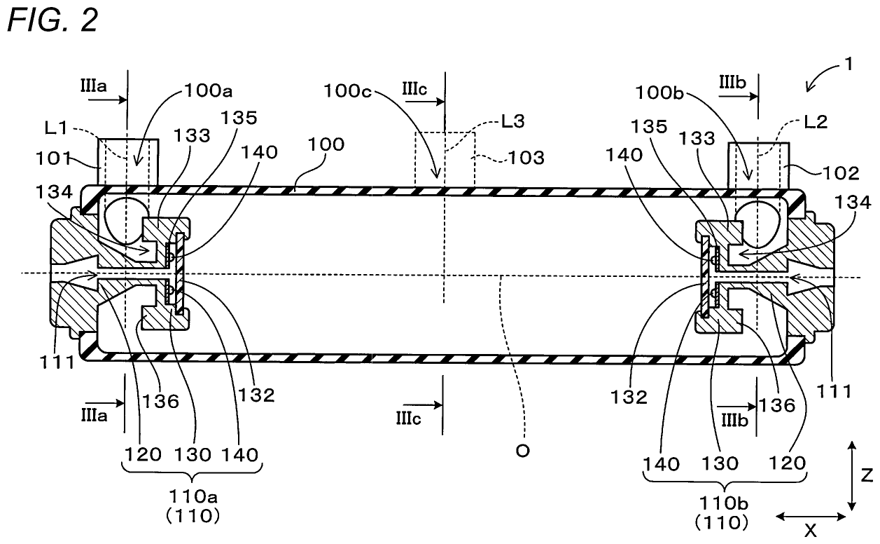

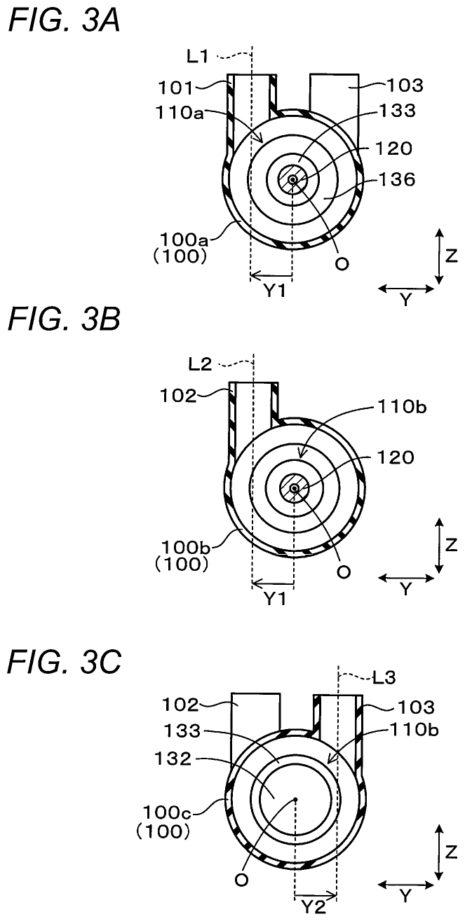

As illustrated in FIG. 1, FIG. 2, and FIG. 3A, the first inlet 101 is provided on the side wall of the first end 100a of the flow path tube 100. As illustrated in FIG. 3A, the first inlet 101 is disposed such that a first central axis L1, which is a central axis of the first inlet 101, does not intersect with the central axis O of the flow path tube 100 and is at a position shifted from the central axis O in a Y1 direction, when viewed from an extending direction of the central axis O of the flow path tube 100. That is, the first inlet 101 is disposed such that an inflow direction of the fluid flowing from the first inlet 101 is offset in the Y1 direction with respect to the central axis O of the flow path tube 100.

Further, as illustrated in FIG. 1, FIG. 2, and FIG. 3B, the second inlet 102 is provided on the side wall of the second end 100b of the flow path tube 100. As illustrated in FIG. 3B, the second inlet 102 is disposed such that a second central axis L2, which is a central axis of the second inlet 102, does not intersect with the central axis O of the flow path tube 100 and is at a position shifted from the central axis O in the Y1 direction, when viewed from the extending direction of the central axis O of the flow path tube 100. That is, similarly to the first inlet 101, the second inlet 102 is disposed such that an inflow direction of the fluid flowing from the second inlet 102 is offset in the Y1 direction with respect to the central axis O of the flow path tube 100.

As illustrated in FIG. 1, FIG. 2, and FIG. 3C, the outlet 103 is provided on the side wall of the central portion 100c between the first inlet 101 and the second inlet 102. As illustrated in FIG. 3C, the outlet 103 is disposed such that a third central axis L3, which is a central axis of the outlet 103, does not intersect with the central axis O of the flow path tube 100 and is at a position shifted from the central axis O in a Y2 direction opposite to the Y1 direction, when viewed from the extending direction of the central axis O of the flow path tube 100. That is, the outlet 103 is disposed such that an outflow direction of the fluid flowing out from the outlet 103 is offset in the Y2 direction with respect to the central axis O of the flow path tube 100.

### 2-2. Configuration of Light Source Unit 110

The light source unit 110 includes the first light source unit 110a provided at the first end 100a of the flow path tube 100 and the second light source unit 110b provided at the second end 100b. As illustrated in FIG. 2, the first light source unit 110a and the second light source unit 110b are disposed to face each other, and the first light source unit 110a is positioned at a position intersecting with the first central axis L1 of the first inlet 101 when viewed from the extending direction of the central axis O of the flow path tube 100 and a direction orthogonal to the first central axis L1. Further, the second light source unit 110b is positioned at a position intersecting with the second central axis L2 of the second inlet 102 when viewed from the extending direction of the central axis O of the flow path tube 100 and a direction orthogonal to the second central axis L2.

As illustrated in FIG. 2, the first light source unit 110a includes the support portion 120, the LED packages 140, and the accommodation portion 130 that accommodates the LED packages 140. Hereinafter, the light source unit 110 provided at the first end 100a will be described, and the second light source unit 110b provided at the second end 100b has the same configuration and the configuration is disposed in a state of being inverted in a longitudinal direction Y of the flow path tube 100.

As illustrated in FIG. 2, the support portion 120 protrudes from the first end 100a of the flow path tube 100 toward the second end 100b, and has a truncated cone-shaped portion. A central axis of the support portion 120 coincides with the central axis O of the flow path tube 100. An inclination angle of a side surface of the truncated cone (angle to a bottom surface) is, for example, 30 to 70°. One end of the support portion 120 on a side with a larger diameter is connected to an end wall of the first end 100a of the flow path tube 100, and one end of the support portion 120 on a side with a smaller diameter is connected to the accommodation portion 130.

A shape of the support portion 120 is not limited to a truncated cone shape, and may be any shape that tapers toward the accommodation portion 130. Although the shape may be tapered in a stepwise manner, it is preferable that the shape is continuously tapered. For example, a truncated pyramid shape may be used. However, in order to form a helical flow, a truncated cone shape is preferable. Further, the entire support portion 120 may not be a truncated cone, and one portion may be a truncated cone and the other portion may be a column. For example, as illustrated in FIG. 1, a side of a front end of the support portion 120 connected to the accommodation portion 130 may have a cylindrical column shape, and the other portion may have a truncated cone shape.

The accommodation portion 130 is connected to the front end of the support portion 120. The accommodation portion 130 accommodates the LED packages 140. The accommodation portion 130 includes a glass plate 132, a base portion 133, and a substrate 135.

The base portion 133 has a cylindrical column-shaped box shape whose upper surface is opened, and an outside bottom surface is connected to the front end of the support portion 120. The substrate 135 is disposed on a bottom surface inside the box, and the LED packages 140 are mounted on the substrate 135. The glass plate 132 is provided on an upper surface of the box and seals an inside of the box. The glass plate 132 is made of a material that transmits the ultraviolet light from the LED packages 140, such as quartz or sapphire. A photocatalytic film that transmits the ultraviolet light may be provided on a surface of the glass plate 132 to inhibit growth of bacteria and prevent organic contamination on the glass plate 132. The glass plate 132 is not limited to a flat plate, and may be lenticular. For example, the glass plate 132 may be a TIR lens, a fly-eye lens, and a Fresnel lens.

As illustrated in FIG. 2, the base portion 133 is formed to extend from the front end of the support portion 120 to an outside of the support portion 120 in the radial direction over the entire periphery of the front end of the support portion 120. Therefore, an inner surface of the base portion 133 is in contact with the flow path space except for a region connected to the support portion 120.

The base portion 133 includes a peripheral wall 136 that protrudes toward the first end 100a in an outer peripheral region of the inner surface thereof, and includes a recess 134 surrounded by the inner surface of the base portion 133 and the peripheral wall 136. The peripheral wall 136 does not need to be provided over the entire peripheral, and may be partially provided. If the peripheral wall 136 is provided over the entire peripheral, air may be accumulated in the recess 134 and the cooling efficiency may be reduced. Further, it is preferable that the peripheral wall 136 is provided outside the LED packages 140 when viewed from the extending direction of the central axis O of the flow path tube 100. That is, it is preferable that the LED packages 140 are positioned in a region of the recess 134. The base portion 133 may be more efficiently cooled.

In Embodiment 1, the base portion 133 has a cylindrical column-shaped box shape, and any shape may be used as long as it has a box shape. For example, the base portion 133 may be a square prism-shaped box shape (square shape). However, from a viewpoint of generating a helical flow, it is preferable to form a cylindrical column-shaped box as that in Embodiment 1.

A material of the support portion 120 and the base portion 133 is preferably a metal material having high thermal conductivity such as SUS or aluminum. Further, titanium may also be used and a surface thereof oxidized to form a photocatalytic film. The propagation of bacteria on the support portion 120 and the base portion 133 may be inhibited.

The LED packages 140 are mounted on the substrate 135. A plurality of LED packages 140 may be mounted. Two LED packages 140 are mounted in FIG. 2. The LED package 140 includes an LED, a substrate on which the LED is mounted, and a lens that seals the LED.

The LED is a light emitting element that emits ultraviolet light. A wavelength of the ultraviolet light is preferably 250 to 285 nm, which is a wavelength having high sterilization efficiency. A plurality of LEDs may be provided in one LED package 140.

It is preferable that the LED package 140 is mounted in a region outside the support portion 120 when viewed from the extending direction of the central axis O of the flow path tube 100. Since the fluid can be brought into contact with a region of the inner surface of the base portion 133 directly below the LED package 140, the accommodation portion 130 may be efficiently cooled.

In Embodiment 1, the packaged LED package 140 is mounted on the substrate 135. The LED may be directly mounted on the substrate 135.

A through continuous hole 111 is provided at a center of the support portion 120 and the accommodation portion 130. The hole 111 is a hole through which a wiring cable that supplies power to the LED package 140 and circuit components on a mounting substrate is inserted. The wiring cable is drawn into the mounting substrate through the hole.

### 3. Regarding Flow Path of Fluid

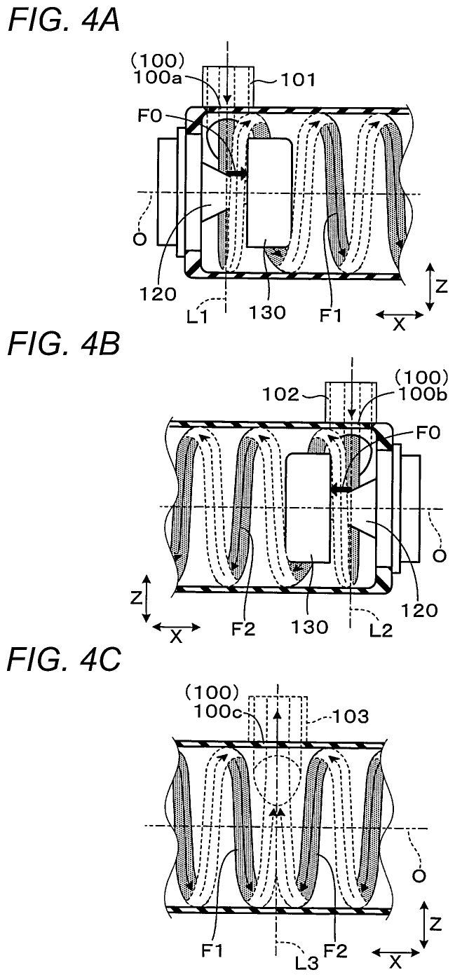

Next, a flow path of the fluid in the flow path space will be described. FIG. 4A is a view schematically illustrating a flow path of a fluid in a vicinity of the first end 100a of the flow path tube 100, and FIG. 4B is a view schematically illustrating a flow path of a fluid in a vicinity of the second end 100b of the flow path tube 100. As illustrated in FIG. 4A, the fluid flowing into the flow path space in the flow path tube 100 from the first inlet 101 hits a side surface of the truncated cone-shaped portion of the support portion 120 and is reflected in an axial direction of the support portion 120 because the side surface is inclined, forming a flow path toward the accommodation portion 130 as indicated by an arrow F0. Therefore, the fluid may be efficiently brought into contact with the accommodation portion 130, and the cooling efficiency may be improved.

Further, since the base portion 133 is formed to extend from the front end of the support portion 120 to the outside of the support portion 120 in the radial direction along the entire periphery of the front end of the support portion 120, the fluid may be brought into contact with the inner surface of the base portion 133. In particular, the fluid is brought into contact with the region of the inner surface of the base portion 133 which is directly below the LED package 140. Therefore, the base portion 133 may be efficiently cooled.

Further, since the peripheral wall 136 is provided on the inner surface of the base portion 133 and the recess 134 surrounded by the peripheral wall 136 exists, the fluid easily remains on the inner surface of the base portion 133. Therefore, heat may be efficiently conducted from the inner surface of the base portion 133 to the fluid, and the cooling efficiency may be improved.

Since the first inlet 101 is offset with respect to the central axis O of the flow path tube 100 as illustrated in FIG. 3A, the flow path that goes around the support portion 120 is formed as illustrated in FIG. 4A. Further, since the support portion 120 is shaped to be tapered toward the second end 100b, the fluid goes around the support portion 120 and toward the extending direction of the central axis O of the flow path tube 100. Therefore, a first helical flow F1 is formed in the flow path space. Since the first helical flow F1 flows in a helical manner, residence time of the fluid in the flow path space is extended, and irradiation time of the ultraviolet light to the fluid is extended, thereby improving the sterilization efficiency.

Further, since the second inlet 102 is offset with respect to the central axis O of the flow path tube 100 as illustrated in FIG. 3B, the flow path that goes around the support portion 120 is formed as illustrated in FIG. 4B. Further, since the support portion 120 is shaped to be tapered toward the first end 100a, the fluid goes around the support portion 120 and toward the extending direction of the central axis O of the flow path tube 100. Therefore, a first helical flow F2 is formed in the flow path space. Since the second helical flow F2 also flows in a helical manner, the residence time of the fluid in the flow path space is extended, and the irradiation time of the ultraviolet light to the fluid is extended, thereby improving the sterilization efficiency.

As illustrated in in FIGS. 3A and 3B, the Y1 direction, which is an offset direction of the first central axis L1 of the first inlet 101 with respect to the central axis O of the flow path tube 100, and the Y1 direction, which is an offset direction of the second central axis L2 of the second inlet 102, are the same direction. Accordingly, the first helical flow F1 and the second helical flow F2 have the same swirling direction, and travel in directions opposite to each other.

Then, as illustrated in FIG. 4C, the first helical flow F1 and the second helical flow F2 join in a vicinity of the central portion 100c of the flow path tube 100, and are discharged from the outlet 103 (see FIG. 3C) offset in the Y2 direction with respect to the central axis O of the flow path tube 100.

### 4. Operations and Effects

As described above, according to the fluid sterilization device 1 in Embodiment 1, since both the first light source unit 110a and the second light source unit 110b are positioned in the flow path space, heat dissipation is promoted by bringing into contact with the fluid flowing through the flow path space, and the cooling effect is improved. Therefore, the first light source unit 110a and the second light source unit 110b are prevented from being damaged even when continuously used. Further, since there is no need to provide a separate cooling device for cooling the first light source unit 110a and the second light source unit 110b, an increase in size and cost may be prevented.

Further, in Embodiment 1, the first inlet 101 is provided on the side wall of the flow path tube 100 at the first end 100a, and the first central axis L1, which is the central axis of the first inlet 101, does not intersect with the central axis O of the flow path tube 100 when viewed from the extending direction of the central axis O of the flow path tube 100. Accordingly, the fluid flowing from the first inlet 101 forms the first helical flow F1 along an inner circumferential surface of the flow path tube 100. Further, the second inlet 102 is provided on the side wall of the flow path tube 100 at the second end 100b, and the second central axis L2, which is the central axis of the second inlet 102, does not intersect with the central axis O of the flow path tube 100 when viewed from the extending direction of the central axis O of the flow path tube 100. Accordingly, the fluid flowing from the second inlet 102 forms the second helical flow F2 in the same swirling direction as the first helical flow F1 along the inner circumferential surface of the flow path tube 100.

By forming the first helical flow F1 and the second helical flow F2 as described above, the fluid flowing from the first inlet 101 into the flow path space and the fluid flowing from the second inlet 102 into the flow path space flow through the flow path space along the helical flows in the same swirling direction, and by smoothing the flow of fluid in the flow path space while lengthening the residence time in the flow path space of the fluid flowing through the flow path space, the cooling effect of the first light source unit 110a and the second light source unit 110b may be improved, thereby improving the sterilization effect of the fluid and optimizing the sterilization effect for continuous use.

Further, in Embodiment 1, the flow path tube 100 has a straight tube shape, the first light source unit 110a and the second light source unit 110b are disposed to face each other, and the first light source unit 110a is positioned at a position intersecting with the first central axis L1 of the first inlet 101 when viewed from the extending direction of the central axis O of the flow path tube 100 and the direction orthogonal to the first central axis L1. Further, the second light source unit 110b is positioned at a position intersecting with the second central axis L2 of the second inlet 102 when viewed from the extending direction of the central axis O of the flow path tube 100 and the direction orthogonal to the second central axis L2. Accordingly, since the fluid can be brought into contact with the first light source unit 110a immediately after flowing into the flow path space from the first inlet 101, and can be brought into contact with the second light source unit 110b immediately after flowing into the flow path space from the second inlet 102, the cooling effect of the first light source unit 110a and the second light source unit 110b may be further improved.

Further, in Embodiment 1, the first light source unit 110a includes the LED package 140 including the light emitting element that emits the ultraviolet light, the support portion 120 provided to protrude from the end surface inside the flow path space at the first end 100a of the flow path tube 100 toward the second end 100b, and the accommodation portion 130 that is provided at the front end of the support portion 120 and accommodates the LED package 140 including the light emitting element. The accommodation portion 130 is formed to extend from the front end of the support portion 120 to the outside of the support portion 120 in the radial direction over the entire periphery of the front end of the support portion 120. Further, the second light source unit 110b also has the same configuration as the first light source unit 110a, and the configuration is reversed in the direction facing the first light source unit 110a (longitudinal direction X).

With this configuration, since the accommodation portion 130 is formed to extend from the front end of the support portion 120 to the outside of the support portion 120 in the radial direction over the entire periphery of the front end of the support portion 120 in the first light source unit 110a and the second light source unit 110b, the fluid may be brought into contact with the inner surface of the accommodation portion 130 (surface closer to the support portion 120), and the accommodation portion 130 may be efficiently cooled.

Further, in Embodiment 1, the accommodation portion 130 in each of the first light source unit 110a and the second light source unit 110b includes the peripheral wall 136 that is formed on the outer surface of the accommodation portion 130 closer to the support portion 120, protrudes toward the support portion 120, and surrounds at least a portion of the front end of the support portion 120. Accordingly, in the first light source unit 110a and the second light source unit 110b, the fluid may be retained on the inner surface of the accommodation portion 130 (surface closer to the support portion 120) by the peripheral wall 136, and the accommodation portion 130 may be more efficiently cooled by the fluid.

Further, in Embodiment 1, the peripheral wall 136 in each of the first light source unit 110a and the second light source unit 110b is positioned outside the LED package 140 when viewed from the central axis direction of the support portion 120. Accordingly, in the first light source unit 110a and the second light source unit 110b, the fluid may be retained in the region of the inner surface of the accommodation portion 130 immediately below the LED package 140, and the accommodation portion 130 may be more efficiently cooled by the fluid.

Further, in Embodiment 1, the support portion 120 in each of the first light source unit 110a and the second light source unit 110b has a truncated cone-shaped portion whose diameter decreases toward the front end of the support portion 120 in the protruding direction (that is, the longitudinal direction X). Accordingly, the fluid flowing into the flow path space in the flow path tube 100 from the first inlet 101 and the second inlet 102 hits the side surface of the support portion 120 having an inclination in the truncated cone-shaped portion and is reflected in the protruding direction of the support portion 120, forming a flow path toward the accommodation portion 130. Therefore, the fluid may be efficiently brought into contact with the accommodation portion 130, and the cooling efficiency may be improved. Further, heat of the accommodation portion 130 may be efficiently diffused to the support portion 120, and the support portion 120 may be efficiently cooled by the fluid.

As described above, according to the above embodiment, the fluid sterilization device 1 may be provided, which improves the cooling effect and prevents an increase in size and cost.

### Embodiment 2

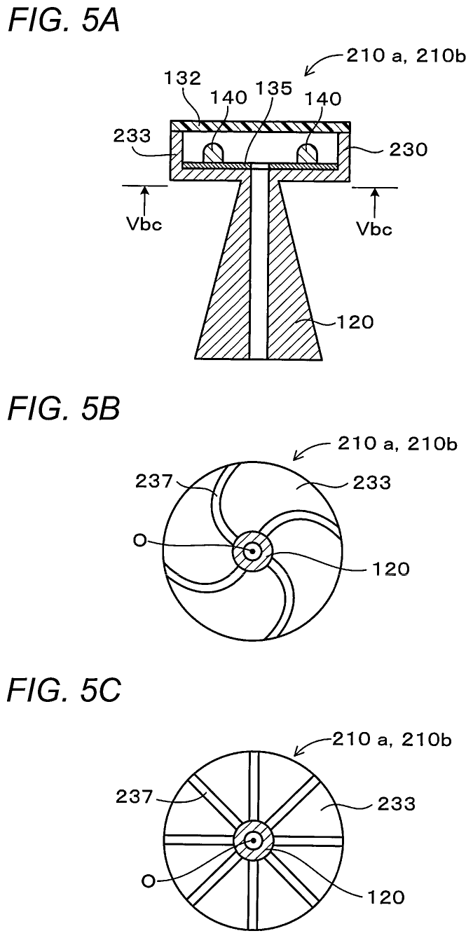

FIGS. 5A, 5B and 5C are views schematically illustrating a configuration of each of the first light source unit 210a and the second light source unit 210b of the fluid sterilization device 1 according to Embodiment 2. As illustrated in FIG. 5A, the first light source unit 210a and the second light source unit 210b each include a support portion 220 and an accommodation portion 230. The support portion 220 has a conical shape whose diameter decreases toward a front end thereof (connection portion with the accommodation portion 230) in the protruding direction along an entire region. The accommodation portion 230 has a configuration in which the base portion 133 of the accommodation portion 130 according to Embodiment 1 is replaced with a base portion 233, and other configurations are similar to that of the accommodation portion 130. The base portion 233 has a configuration in which the peripheral wall 136 is eliminated from the base portion 133, and an outer peripheral region on an inner surface of the base portion 233 is flat.

In Embodiment 2, the effect of retaining the fluid on the inner surface of the accommodation portion 230 is not obtained by the peripheral wall 136, whereas other effects may be obtained in the same manner as in Embodiment 1. Further, since the entire region of the support portion 220 has a conical shape, a flow path toward the accommodation portion 230 is more easily formed. Therefore, the fluid can be efficiently brought into contact with the accommodation portion 230, and the cooling efficiency may be further improved.

In Embodiment 2, in the first light source unit 210a and the second light source unit 210b, a groove 237 may be provided on the inner surface of the accommodation portion 230 (inner surface of the base portion 233), and the fluid may be guided from a center side to an outer periphery side of the inner surface of the accommodation portion. Alternatively, a wall-shaped protrusion portion may be provided instead of the groove 237.

FIGS. 5B and 5C are each a sectional view illustrating a cross section taken along line Vbc-Vbc in FIG. 5A. FIG. 5B illustrates a case in which a spiral groove 237 is provided on the inner surface of the base portion 233. A center of the spiral is a center of the support portion 220. By providing such spiral groove 237, contact time between the fluid and the accommodation portion 230 is increased, so that the accommodation portion 230 may be efficiently cooled. Further, a flow path swirling toward the outer periphery of the inner surface of the accommodation portion 230 may be formed, and the fluid passing between an inner wall of the flow path tube 100 and the accommodation portion 230 easily forms a helical flow.

FIG. 5C illustrates a case in which a radial groove 237 is provided on the inner surface of the base portion 233. By providing such groove 237, the fluid may be guided to the outer periphery, and the fluid may smoothly pass between the inner wall of the flow path tube 100 and the accommodation portion 230. Further, since the contact time between the fluid and the accommodation portion 230 is increased, the accommodation portion 230 may be efficiently cooled.

### Embodiment 3

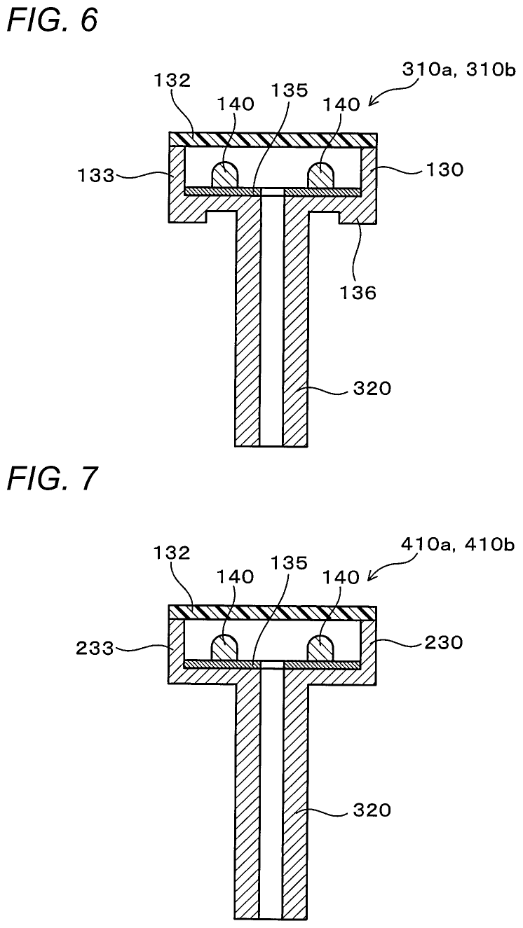

FIG. 6 is a view schematically illustrating a configuration of each of the first light source unit 310a and the second light source unit 310b of the fluid sterilization device 1 according to Embodiment 3. The first light source unit 310a and the second light source unit 310b each include a support portion 320 and an accommodation portion 130. As illustrated in FIG. 6, the support portion 320 has a cylindrical column shape. Other configurations are the same as those of the support portion 120 according to Embodiment 1. The accommodation portion 130 is the same as that in Embodiment 1.

In Embodiment 3, since the support portion 320 is a cylindrical column, there is no effect of causing the fluid to travel toward the accommodation portion 130, whereas other effects may be obtained in the same manner as in Embodiment 1.

### Embodiment 4

FIG. 7 is a view schematically illustrating a configuration of each of a first light source unit 410a and a second light source unit 410b of a fluid sterilization device 1 according to Embodiment 4. The first light source unit 410a and the second light source unit 410b each include the support portion 320 and the accommodation portion 230. The support portion 320 is the same as the support portion 320 in Embodiment 3, and has a cylindrical column shape. The accommodation portion 230 has a configuration similar to that of the accommodation portion 230 in Embodiment 2, and the configuration includes no peripheral wall 136 in the outer peripheral region of the inner surface of the base portion 233.

In Embodiment 4, there is no effect of causing the fluid to travel toward the accommodation portion 230 or the effect of causing the fluid to retain on the inner surface of the accommodation portion 230, whereas other effects may be obtained in the same manner as in Embodiment 1.

In Embodiment 4, similarly to FIGS. 5B and 5C in Embodiment 2, a groove may be provided on the inner surface of the accommodation portion 230, and the fluid may be guided from the center side to the outer peripheral side of the inner surface of the accommodation portion.

### Modification 1 of Embodiment 1

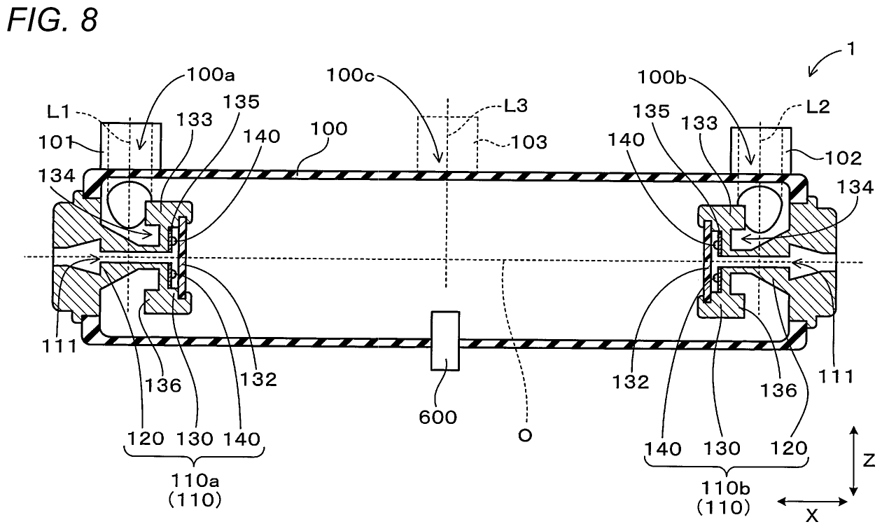

FIG. 8 is a view schematically illustrating a configuration of a fluid sterilization device 1 according to Modification 1 of Embodiment 1. As illustrated in FIG. 8, the fluid sterilization device 1 according to Modification 1 includes a light intensity sensor 600 in the central portion 100c of the flow path tube 100. Other configurations are the same as those of the fluid sterilization device 1 according to Embodiment 1.

The light intensity sensor 600 is a sensor that detects an intensity of the ultraviolet light in the central portion 100c in the flow path tube 100. For example, outputs of the first light source unit 110a and the second light source unit 110b are controlled such that the intensity of the ultraviolet light in the central portion 100c is equal to or greater than a predetermined value or more.

Further, the light intensity sensor 600 also serves as a rectifying plate. The light intensity sensor 600 is a wall-shaped protrusion that is provided on the inner wall of the flow path tube 100 and protrudes toward the central axis of the flow path tube 100. The light intensity sensor 600 has a wall shape along a direction of each of the first helical flow F1 and the second helical flow F2, thereby allowing both helical flows to be maintained in the vicinity of the central portion 100c of the flow path tube 100.

The present disclosure is not limited to the above-described embodiments, and may be applied to various embodiments without departing from the gist of the present disclosure.

What is claimed is:

1. A fluid sterilization device comprising:
a cylindrical flow path tube having a flow path space therein through which a fluid is capable of flowing;
a first inlet provided at a first end of the flow path tube and allowing the fluid to flow into the flow path space;
a second inlet provided at a second end of the flow path tube opposite to the first end and allowing the fluid to flow into the flow path space;
an outlet provided between the first end and the second end of the flow path tube and allowing the fluid flowing through the flow path space to flow out;
a first light source unit disposed at the first end within the flow path space and configured to emit ultraviolet light into the flow path space; and
a second light source unit disposed at the second end within the flow path space and configured to emit ultraviolet light into the flow path space.

2. The fluid sterilization device according to claim 1, wherein
the first inlet is provided on a side wall of the flow path tube at the first end, and a first central axis, which is a central axis of the first inlet, does not intersect with a central axis of the flow path tube when viewed from an extending direction of the central axis of the flow path tube, such that a fluid flowing from the first inlet forms a first helical flow along an inner circumferential surface of the flow path tube, and
the second inlet is provided on a side wall of the flow path tube at the second end, and a second central axis, which is a central axis of the second inlet, does not intersect the central axis of the flow path tube when viewed from the extending direction of the central axis of the flow path tube, such that a fluid flowing from the second inlet forms a second helical flow in the same swirling direction as the first helical flow along the inner circumferential surface of the flow path tube.

3. The fluid sterilization device according to claim 2, wherein
the flow path tube has a straight tube shape,
the first light source unit and the second light source unit are disposed to face each other,
the first light source unit is positioned at a position intersecting with the first central axis when viewed from the extending direction of the central axis of the flow path tube and a direction orthogonal to the first central axis of the first inlet, and
the second light source unit is positioned at a position intersecting with the second central axis when viewed from the extending direction of the central axis of the flow path tube and a direction orthogonal to the second central axis of the second inlet.

4. The fluid sterilization device according to claim 1, wherein
the first light source unit includes
a light emitting element that emits ultraviolet light,
a support portion provided to protrude from an end surface of the first end inside the flow path space toward the second end, and
an accommodation portion that is provided at a front end of the support portion and accommodates the light emitting element,
the accommodation portion is formed to extend from the front end of the support portion to an outside of the support portion in a radial direction over an entire periphery of the front end of the support portion, and
the second light source unit has the same configuration as the first light source unit, and the configuration is reversed in a direction facing the first light source unit.

5. The fluid sterilization device according to claim 4, wherein
the accommodation portion in each of the first light source unit and the second light source unit has a peripheral wall that is formed on an outer surface of the accommodation portion closer to the support portion, protrudes toward the support portion, and surrounds at least a portion of the front end of the support portion.

6. The fluid sterilization device according to claim 5, wherein
the peripheral wall in each of the first light source unit and the second light source unit is positioned outside the light emitting element when viewed from a central axis direction of the support portion.

7. The fluid sterilization device according to claim 4, wherein
the support portion in each of the first light source unit and the second light source unit has a truncated cone-shaped portion whose diameter decreases toward the front end of the support portion in a protruding direction.

8. The fluid sterilization device according to claim 4, wherein
the support portion in each of the first light source unit and the second light source unit has a cylindrical column shape.

9. The fluid sterilization device according to claim 4, wherein
a surface of the accommodation portion in each of the first light source unit and the second light source unit closer to the support portion is provided with a spiral groove or a protrusion portion.

10. The fluid sterilization device according to claim 4, wherein
a surface of the accommodation portion in each of the first light source unit and the second light source unit closer to the support portion is provided with a radial groove or a protrusion portion.
